(19) United States
(12) Patent Application Publication
(10) Pub. No.: US 2010/0028293 A1
(43) Pub. Date: Feb. 4, 2010

(54) Agents for control of codling moth in fruit orchards

(75) Inventors: Oded Shoseyov, Carmei Yosef (IL); Shu Wei, Saskatoon (CA); Israel Fluksman, Tel Mond (IL)

Correspondence Address: BROWDY AND NEIMARK, P.L.L.C.;624 NINTH STREET, NW, SUITE 300, WASHINGTON, DC, 20001-5303, US

(73) Assignees: Efal Chemical Industries Ltd., Netanya (IL); Yissum Research Industries Development Company of the Hebrew University of Jerusalem, Jerusalem (IL)

(21) Appl. No.: 11/665,068

(22) PCT Filed: Oct. 11, 2005
(86) PCT No.: PCT/IL05/01079

§ 371 Date: Oct. 5, 2009

Related U.S. Application Data
(60) Provisional application No. 60/617,679, filed on Oct. 13, 2004.

(51) Int. Cl. A01N 31/02; C07C 33/02; A01P 17/00; A01P 19/00; C12Q 1/34

(52) U.S. Cl. 424/84; 568/909.5; 514/739; 435/18

(57) ABSTRACT

The invention provides a method for identifying volatile insect repellents and/or attractants released from their non-volatile glycosides by the action of -glucosidase on plant tissue extracts. By this method, geraniol was identified as codling moth repellent and methyl salicylate, (2R,5R)-theaspirane, and (2S,5R)-theaspirane, and to a lesser extent linalool and benzyl alcohol as codling moth attractants. The invention provides compositions comprising said repellent and attractants and methods for control of codling moth in fruit, preferably apple, orchards.

### FIELD OF THE INVENTION

The present invention relates to a method for identification of insect relents and attractants and to the use of certain insect repellent and attractants for integrated control of codling moth in fruit orchards.

### BACKGROUND OF THE INVENTION

The codling moth larva is a very destructive pest. Codling moth adults are small with a wing span of ½ to ¾ inch, about the size of a house fly. Their gray mottled appearance blends well with bark, making them difficult to detect. If the adults are being trapped, codling moths can be distinguished from other moths associated with fruit trees by their dark brown wing tips that have shiny, coppery markings. Eggs are laid singly on fruit, nuts, or nearby leaves. In pears, eggs may also be laid at the base of leaf clusters. On apples and pears, larvae penetrate fruit and bore into the core leaving brown-colored holes in the fruit that are filled with frass (larval droppings). In plums, and occasionally other stone fruits, codling moth bores into the fruit all the way to the pit.

The codling moth (CM) Cydia pomonella (L.) is the most economically important insect for apple cultivation worldwide. It has a close ecological association with apples as well as some other plants (Shel'deshova, 1967). Bengtsson et al. observed the behavioural response of codling moth adults to apple branches with or without green fruit in the wind tunnel and found that both apple green fruit and leaves alone attract codling moth females (Bengtsson et al., 2001). This association has been known to be largely mediated by some volatile attractants released from the host plants (Bengtsson et al., 2001; Sutherland and Hutchins, 1972; Yan et al., 1999; Knight and Light, 2001; Light et al., 2001). (E,E)-α-farnesene (Sutherland and Hutchins, 1972; Wearing and Hutchins, 1973), linalool, and β-caryophyllene (Bengtsson et al., 2001) have been identified as attractants for codling moth adults or neonate larvae (Knight and Light, 2001; Light et al., 2001). However some of these compounds are also present in the volatile profiles of a wide variety of non-host plants. It is therefore reasonable to assume that altering the volatile composition of the host plants may change their association with codling moth insects.

It is well known that many plants contain significant amounts of glycosidically bound volatiles. Usually, plant leaves show the highest variability of aglycones, followed by flowers, stems, and roots (Stahl-Biskup et al., 1993). Some glycosidically bound volatile compounds in apple leaves and fruit have been isolated and identified (Schwab and Schreier, 1988; Stingl et al., 2002). Furthermore, successful aroma enhancement of fruit juices and wines by exogenous application of β-glucosidase has been demonstrated (Belancic et al., 2003; Shoseyov et al., 1990; Yanai and Sato, 1999).

Codling moth can be very difficult to manage, especially if its population has been allowed to build up over a season or two. Where populations are high and many infested trees are nearby, insecticide applications may be necessary to bring populations down to very low levels. However, insecticides are very difficult to time accurately, and the only highly effective material available is toxic to natural enemies and honeybees. Nonchemical methods are therefore preferred. Control of codling moth in commercial orchards can rely on nonchemical tools including regular examination of the trees and fruit (termed scouting), pheromone trapping, and the use of weather monitoring and degree-day models. It would be very desirable to find natural non-toxic compounds that could be useful against codling moth in fruit orchards.

Geraniol, an olefinic terpene alcohol found in many essential oils such as oil of rose, oil of palmarosa, citronella, lemon grass, etc., is described in The Merck Index (13^{th} Edition, 2001, #4415) as insect attractant. However, several publications describe geraniol as a volatile insect repellent.

U.S. Pat. No. 4,774,081 discloses geraniol as an example of a contact insect repellent against cockroaches and other crawling insects. Chem.Abstracts, Vol. 105:204742q, discloses that compounds from the leaves of bay (Laurus nobilis L.) are useful repellents for Tribolium castaneum (Herbst) and these compounds include, inter alia, geraniol when present at 50 ppm. Chem. Abstracts, Vol. 113:110945w (abstract of JP 02/67202) discloses that, inter alia, linalool, geraniol, citronellol and nerol are repellent when incorporated into porous inorganic microcapsules against cockroaches, slugs, ants, etc.

U.S. Pat. No. 5,227,406, U.S. Pat. No. 5,346,922, U.S. Pat. No. 5,648,398 and U.S. Pat. No. 5,621,013 disclose an insect repellent for humans and animals for repelling ticks carrying Lyme disease, as well as biting flies and triatomes (Chagas bugs), said repellent comprising terpineol, citronella, and one or both of rhodinol extra and geraniol as actives provided in a conveying medium. The actives are used in small percentages, e.g. as little as 0.01%, preferably at between 0.05% and 0.08%, and preferably less than 1%, yet are synergistically efficacious, particularly against ticks carrying Lyme disease.

U.S. Pat. No. 5,633,236 describes a method of repelling Musca domestica L.(Diptera:Muscidae), Aedes aegypti, Culex nigripalpus, Aedes atlanticus, Culex salinarius, Aedes vexans, Culex spp., Simulium spp., Psoroferia ferox, Aedes infirmatus, Drosophila melanogaster, Coccinellidae, Anopheles crucians, Psoroferia columbiae, Culicoides spp., and Aedes spp., for a finite period of time, consisting essentially of the step of exposing a three-dimensional space to an effective repelling concentration and quantity of geraniol or a geraniol precursor-containing composition consisting essentially of 50-100% geraniol or a geraniol precursor (e.g., geranyloxy-1,3,2-dioxaborinane, digeranyloxydimethylsilane and geranyl glycosides, for example, geranyl 6-O-(alpha-L-rhamnopyranosyl)-beta-D-glucopyranoside) with the remainder of the composition (if indeed the composition is not 100% geraniol) being a compound selected from the group consisting of citronellol and nerol. The above-stated repellents are described to be useful as such or contained in a polymer which can be a biodegradable polymer such as compositions containing a major proportion of poly(epsilon caprolactone) homopolymers. U.S. Pat. No. 5,401,500, U.S. Pat. No. 6,143,288, U.S. Pat. No. 5,635,173 and U.S. Pat. No. 5,665,781 describe such a method of repelling Musca domestica L.(Diptera:Muscidae) and/or Aedes aegyptae comprising exposing to a geraniol-containing composition consisting essentially of 50-100% of geraniol with the remainder of the composition being a compound selected from the group consisting of citronellol and nerol.

U.S. Pat. No. 5,753,686 describes a method for repelling at least one of the insect species: (i) Haematobia irritans (Linnaeus) (commonly known as the horn fly); and (ii) Solenopsis invicta Buren (commonly known as the “red imported fire ant”) from a surface or volume inhabited by at least one of said insect species consisting of the step of applying to said surface or said volume a “red imported fire ant” and/or horn fly-repelling quantity and concentration of a geraniol-containing mixture comprising: (i) from 0 up to about 20% by weight of nerol; (ii) from about 20 up to about 40% by weight of citronellol; and (iii) from about 50 up to about 70% by weight of geraniol which geraniol-containing mixture is defined according to specific GLC profiles set forth in the figures and refractive index and density set forth in the specification of said patent.

Products containing geraniol are available commercially in the form of bands, towelettes and a pump spray bottle, for use as insect repellent. By spraying the liquid directly on clothing or skin, the geraniol vapors form a protective barrier to deter blood-sucking insects from biting. These products have proven to be effective against mosquitoes, fleas, ticks, black flies, gnats, chiggers, no-see-ums, and many others.

As far as known to the present inventors, geraniol has not been described in the literature as repellent to codling moth.

### SUMMARY OF THE INVENTION

In one aspect, the present invention relates to a method for identifying volatile insect repellents and/or attractants released from their non-volatile glycosides by the action of β-glucosidase on plant tissue extracts.

By application of this method in apple leaf extracts, geraniol was identified as codling moth repellent and methyl salicylate, the (2R,5R)- and (2S,5R)-aspirane isomers, linalool and benzyl alcohol, were identified as codling moth attractants.

Thus, in another aspect, the present invention relates to compositions comprising geraniol for use as codling moth repellents, and to methods for repelling codling moth in fruit orchards by treating the orchards with said geraniol repellent composition.

In a further aspect, the present invention relates to compositions comprising methyl salicylate, an aspirane isomer, linalool, benzyl alcohol, or a combination thereof, for use as codling moth attractants, and to methods for attracting codling moth in fruit orchards by treating the orchards with said attractant composition.

In yet another aspect, the present invention relates to a method for integrated control of codling moth in fruit orchards by treating the orchards with both said geraniol repellent and attractant compositions.

### BRIEF DESCRIPTION OF THE FIGURES

FIG. 1 shows attraction of apple leaf extracts treated with recombinant β-glucosidase (BGL1) and glucoimidazole (β-glucosidase inhibitor). Leaf BGL1-traps with leaf extract treated with β-glucosidase; Leaf Inhi—traps with the leaf extract treated with glucoimidazole; Leaf—traps with leaf extract; Water—trap with water. The same lowercase and uppercase letter indicated no significant difference in the number of the insects caught in the different traps and around the traps respectively, p=0.05, multiple Turkey test.

FIGS. 2A-2B show increased levels of compounds released from apple (cv. Anna) leaf extracts added with external β-glucosidase. (2A) GC chromatographs of apple leaf extracts added with β-glucosidase (BGL1), glucoimidazole (inhibitor), or nothing. Peak 1, benzyl alcohol; 2, linalool; 3, methyl salicylate; 4, geraniol; 5, (2R,5R)-theaspirane; 6, (2S,5R)-theaspirane. (2B) Increased levels of compounds in the leaf extracts with β-glucosidase BGL1. The same letter indicated no significant difference in the number of the insects caught in the different traps, p=0.05, multiple Turkey test.

FIG. 3 shows attraction/repulsion effect of β-glucosidase-enhanced compounds on codling moth insects. Trapping tests were conducted in screen cages with paired traps and 60 adult insects. The numbers of insects caught in the traps were recorded and analyzed by t-test (p<0.05). +G, with addition of geraniol at a concentration of 39.4 ng ml⁻¹; Mixed, mixture of all the attractants at the concentrations detected in leaf extracts.

### DETAILED DESCRIPTION OF THE INVENTION

In one aspect, the present invention relates to a method for identifying volatile insect repellents and/or attractants released from non-volatile glycosides by the action of β-glucosidase on plant tissue extracts, comprising:

(i) homogenizing plant tissue and subjecting the plant tissue homogenate to the action of an extraction buffer;

(ii) treating the plant extract with a β-glucosidase or with a β-glucosidase inhibitor;

(iii) identifying by GC-MS specific volatiles released in step (ii) which levels were enhanced in the extracts treated with β-glucosidase relative to the extracts treated with the β-glucosidase inhibitor; and

(iv) testing each one of the volatiles identified in step (iii) for their effect on the behavior of insects,

thus identifying volatiles which are repellents or attractants of each of the tested insects.

Any β-glucosidase can be used in the method of the invention, preferably a recombinant β-glucosidase, and any β-lucosidase inhibitor may be used such as, but not limited to, glucoimidazole.

In the present application, the recombinant Aspergillus niger β-glucosidase (hereinafter BGL1) produced in Pichia pastoris was utilized to hydrolyze the glycosides of volatiles present in the leaves of apple (Malus domestica, cv. Anna). The effect of the enzymatically-released volatile compounds which level was higher in the leaf extract treated with BGL1 compared with the leaf extract treated with glucoimidazole, was evaluated on the behavior of codling moth adults.

According to the invention, glycosidically bound volatiles released from apple leaf extracts (cv. Anna) were analyzed by SPME-GC-MS and their behavioral effects on codling moth (CM) adults were evaluated in cage bioassays. The levels of 1-octanol, linalool, geraniol, benzyl alcohol, methyl salicylate, (2R,5R)-theaspirane and (2S,5R)-theaspirane were significantly increased in the leaf extracts containing the A. niger β-glucosidase (BGL1) compared to the extracts containing the glucoimidazole. The attractiveness of individual compounds to CM adults was found in the following decreasing order: methyl salicylate and mixture of two theaspirane isomers, followed by linalool and benzyl alcohol. Geraniol was found repellent to CM adults. The addition of geraniol (39.4. ng ml⁻¹) to any of the individual volatiles or to a mixture of these attractants eliminated their attractiveness. Our data suggest the possible application of geraniol as a repellent and methyl salicylate or theaspiranes as attractants for the integrated control of CM in apple orchards.

The purpose of some experiments of the present invention was to determine the potential to release plant volatiles in apple leaves by the broad specificity A. niger BGL1, and to determine potential interactions between the released volatiles and apple's most important insect, the codling moth.

It has thus been found, in accordance with the present invention, that geraniol is repellent to codling moth.

The present invention thus relates to compositions comprising geraniol for use as codling moth repellents.

The composition may contain only geraniol, natural or synthetic, or it may comprise other ingredients such as nerol and citronellol, preferably from about 50 up to about 70% by weight of geraniol, from 0 up to about 20% by weight of nerol, and from about 20 up to about 40% by weight of citronellol.

It has further been found, in accordance with the present invention, that methyl salicylate, the (2R,5R) and (2S,5R) theaspirane isomers, linalool and benzyl alcohol are attractants of codling moth.

The present invention thus further relates to compositions comprising an agent selected from the group consisting of methyl salicylate, a theaspirane isomer, linalool, benzyl alcohol, and a combination thereof, for use as attractants of codling moth. These attractants, taken alone or in combination, are useful as bait enhancers for acute toxins and/or trapping devices. In preferred embodiments, the attractant is methyl salicylate, (2R,5R) theaspirane, (2S,5R) theaspirane, or a combination thereof.

Volatile insect repellents and attractants as defined herein have the disadvantage of giving protection only for relatively short periods of time due to their rapid evaporation and/or absorption by the treated substrate. These two factors, absorption and evaporation, lead to the need of frequent applications, which are bothersome and time-consuming. Various materials as known in the art can be used to extend the residual activity of the volatile insect repellents and attractants according to the present invention and all of them are encompassed by the present invention.

The composition comprising geraniol or a codling moth attractant as defined herein may be in combination with natural or synthetic compatible polymers which may or may not be biodegradable. The polymer may be a cellulose derivative including, but not limited to, cellulose ethers, e.g., methyl cellulose, ethyl cellulose, carboxymethyl celluloses, hydroxyethyl celiuloses, hydroxypropyl celluloses; cellulose esters, e.g., acetyl celluloses. The polymer may also be coating materials based on gelatin and other materials, to create beadlets carried and coated with corn starch. The polymer may also be high density polyethylene or low density polyethylene, or biodegradable polymers such as biodegradable thermoplastic polyurethanes, biodegradable ethylene polymers having ester linkages in the main chain, and poly(epsilon-caprolactone) homopolymers as disclosed in U.S. Pat. Nos. 4,496,467; 4,469,613 and 4,548,764, hereby incorporated herein by reference.

The composition of the invention may be sprayable, in which case it also comprises an aqueous diluent, or it may be a concentrate, requiring dilution, dispersion or dissolution in water to provide a sprayable composition.

In one preferred embodiment of the invention, the composition of the invention comprises microcapsules containing geraniol or one or more attractans of the invention. These microcapsules provide several advantages including slow release of the geraniol or of the attractant. In addition, these microcapsules may be small enough to be suitably used in sprayable compositions without blocking spray nozzles. Furthermore, geraniol or the attractant(s) will “glue” and effectively be retained on the foliage (e.g., the leaves or other photosynthesizing organs) or on the bark, of the plants, for example in the form of droplets.

In a further embodiment of the invention, the geraniol or attractant composition can also be incorporated into a granulate that is capable of releasing it slowly and in a controlled manner. Like the above-mentioned microcapsules, the granulate is present in the matrix and is distributed in the form of droplets or droplet-like units. The granulates may consist of small-particled inorganic carriers and/or organic polymers such as those familiar to the person skilled in the art.

The present invention also provides a process for repelling codling moth, which comprises treating an environment infested or imminently capable of being infested with said codling moth with a codling moth repelling composition comprising geraniol.

The present invention further relates to a method of repelling codling moth for a finite period of time in fruit orchards inhabitable by codling moth, said method comprising treating the orchards with a geraniol repellent composition.

In another aspect, the invention relates to a method of attracting codling moth in fruit orchards, said method comprising treating the fruit orchards with an attractant selected from the group consisting of methyl salicylate, a theaspirane isomer, linalool, benzyl alcohol, and a combination thereof.

In one embodiment, the invention provides a method of attracting codling moth in an apple orchard to an insect trap comprising the step of exposing the environment surrounding said trap to an insect attractant containing polymer which consists of a mixture of a polymer and at least 1% by weight of said polymer of an attractant selected from the group consisting of methyl salicylate, a theaspirane isomer, linalool, benzyl alcohol, and a combination thereof.

The invention further relates to a method for the integrated control of codling moth for a finite period of time in fruit orchards inhabitable by codling moth, said method comprising treating the orchards with a geraniol repellent composition and a composition comprising a codling moth attractant selected from the group consisting of methyl salicylate, a theaspirane isomer, linalool, benzyl alcohol, and a combination thereof.

The fruit orchards that can be treated with the geraniol repellent composition and the attractant composition of the invention are those attacked by the codling moth pest and include apple, pear, quince, walnut, hawthorn and crab apple orchards. In one most preferred embodiment, the method of the invention is applied to apple orchards, and the trees are sprayed during the fruiting season.

The invention will now be illustrated by the following non-limiting examples.

### Examples

Materials and Methods

(i) Insects—Eggs of the CM Cydia pomonella L. were hatched at room temperature in a petri dish. The larvae were fed an artificial diet (Manduca Premix-Heliothis Premix, Stonefly Inc., Brvan, Tex.), kept at 25±0.5° C., 60±1% relative humidity and a photoperiod of 16:8 h (L:D) until the fifth instar larvae, which were transferred to corrugated paper strips (2 cm²). Upon emergence, adult insects of mixed sex and age (within 2 days) were used for the cage bioassays.

(ii) Apple leaf extract treatments—Apple (cv. Anna) spurs with both leaves and fruit were excised from a commercial orchard in the suburb of Rehovot, Israel, in June 2004. The leaves were excised from the spurs, rinsed with double distilled water (DDW) and then dried with filter paper. Dried leaves were homogenized in liquid nitrogen. Three times the weight of ice-cold extraction buffer containing 10 mM EDTA and 4 mM DTT in 50 mM citrate buffer, pH 4.3, was added to the leaf homogenate (4-5 g per treatment). The mixture was then rotated for 1 h at 4° C. and centrifuged at 16,000 g for 5 min. The supernatant (10 ml per treatment) was collected. In one treatment, 1 unit of recombinant A. niger BGL1 produced in P. pastoris (Dan et al., 2000) was added and the solution was kept at 37° C. for 4 h. In another treatment, 2 μM glucoimidazole (a β-glucosidase inhibitor) was added to the leaf homogenate immediately after the addition of the extraction buffer in order to block any endogenous β-glucosidase activity. Quantitative β-glucosidase activity was assayed using p-nitrophenyl-β-D-glucopyranoside (pNPG) as the substrate according to Shoseyov et al (1990).

(iii) Volatile collection and GC-MS (gas chromatography (GC) and mass spectrometry (MS)) analysis—Headspace volatiles released from the mixtures of leaf extracts and fruit juices were collected with a solid-phase microextraction (SPME) fiber coated with 100 μm polydimethylsiloxane (PDMS). The fiber was exposed to the headspace volatiles for 30 min. During SPME the leaf extract temperature inside the vial was 60° C. without stirring. The loaded SPME fiber was then desorbed for 3 min in the injection port of a Varian-3 GC-MS system equipped with a 30 m, 0.25 mm ID DB-5 capillary column (J&W, Folson, Calif.). Sampling and desorption times were precisely controlled by a Varian 8200 Autosampler (Varian, Palo Alto, Calif.). 3-Octanol (0.1 ppm) was added to every sample as an internal standard. Each sample was analyzed in independent triplicates. GC-MS parameters were set according to Shalit et al (2003). The injector temperature was 250°, set for splitless injection. The column was set to 50° C. for 1 min and then the temperature was increased to 200° C. at a rate of 4° C. min⁻¹. Mass range was recorded from 45 to 450 mass-to-charge ratio, with electron energy of 70 eV.

Most volatile chemicals were identified by comparing their mass spectra and retention data with those of authentic compounds, supplemented with the Wiley mass spectrum library (McLafferty, 1994) and literature data (Adams, 1995). The volatiles were quantified by calculating the concentrations relative to those of the internal standard. The areas of the volatile component peaks were normalized by the area of the internal standard peak.

(iv) Chemicals—Most synthetic standards were purchased from Sigma/Aldrich (St. Louis, Mo.). Purities ranged from 98 to 99.5%. Mixtures of (2R,5R)-theaspirane and (2S,5R)-theaspirane were from Fluka (Buchs S G, Switzerland).

(v) Cage bioassays—Trapping tests were conducted in screen cages (96×68×45 cm) in the laboratory at room temperature (23° C.±3° C.) based on the method of Zhu et al. (2003). Sixty codling moth adults of mixed sex and ages (2 days after pupal emergence) were released into a cage containing the traps with different treatments. Traps were constructed from 100-ml beakers covered with a white paper lid that had a hole (5 mm diam.) at the center. A Whatman paper wick (10 cm long) was used as a dispenser. To examine the effects of adding recombinant BGL1 to the leaf extract, four traps were constructed and put into the same cage. The first trap contained 10 ml of leaf extract with the addition of recombinant BGL1. The second trap contained the same amount of leaf extract with the addition of glucoimidazole. The third trap contained only leaf extract and the fourth trap contained water. The insects caught in the traps, or around the trap, were counted 12 h after their release. The insects around the trap were defined as follows: the space of the screen cage was divided equally into four parts. Each trap was put in the center of each part. The number of the insects present in each specific space, not including the ones inside the trap, was obtained in the experiments. To test the attractiveness of the synthetic compounds, traps were made with the individual chemicals that were first dissolved in ethanol at the concentrations determined by the SPME-GC-MS analyses in leaf extracts treated with BGL1. The final concentration of ethanol was 0.01%. Each trap was kept in the cage with only a control water trap including 0.01% ethanol. The experiments were conducted overnight (from 9 pm to 9 am) to cover their active time before the twilight in the early morning. Each experiment was replicated five times except with control 1-octanol, which showed no behavioral activity to codling moth in the preliminary experiments. This experiment was replicated 3 times. The traps were randomly arranged in the replications to avoid possible position effect of the traps. The studies on sex ratios of trapped insects were skipped because of complicated involvement of insect pheromones.

### Example 1

### The Effect of β-Glucosidase Activity on the Attraction/Repulsion of Apple Leaf Extract to Codling Moth Adults

Quantitative P-glucosidase activity assay using p-nitrophenyl β-D-glucopyranoside (pNPG) as the substrate confirmed the residual activity of endogenous β-glucosidase in apple leaf extracts (0.24 unit g⁻¹ fresh weight.min⁻¹). Treatment with glucoimidazole (Heightman and Vasella, 1999) resulted in undetectable levels of β-glucosidase activity in the leaf extract.

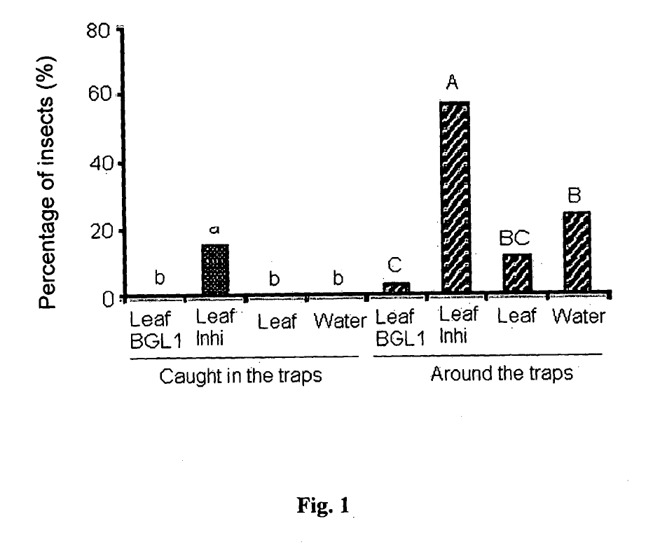

Traps containing apple leaf extract and 2 μM glucoimidazole caught significantly more adult codling moths inside the traps than did the traps containing leaf extract, leaf extract treated BGL1, or water. Additionally, around the leaf plus inhibitor traps, there were significantly more insects (FIG. 1) than any other traps. These well consistent results suggested that aglycones released by β-glucosidase activity have a repellent effect on codling moth insects. Furthermore, the complete inhibition of β-glucosidase activity by glucoimidazole and the significant attraction of leaf extract treated with this β-glucosidase inhibitor indicate that these glycosides and the β-glucosidase are located in different compartments in the intact leaf.

Attraction or orientation responses of phytophagous insects to host plant odor may be enhanced or increased with injury to the plant. The increased response of codling moth larvae and adults to apple fruit infested with other codling moth larvae has been reported (Landolt et al., 2000; Reed and Landolt, 2002). In this study, we demonstrated that crushed apple leaves with glucoimidazole retain their attractiveness to the codling moth adults.

### Example 2

### GC-MS Analysis of Apple Leaf Extracts Treated with BGL1 or β-Glucosidase Inhibitor

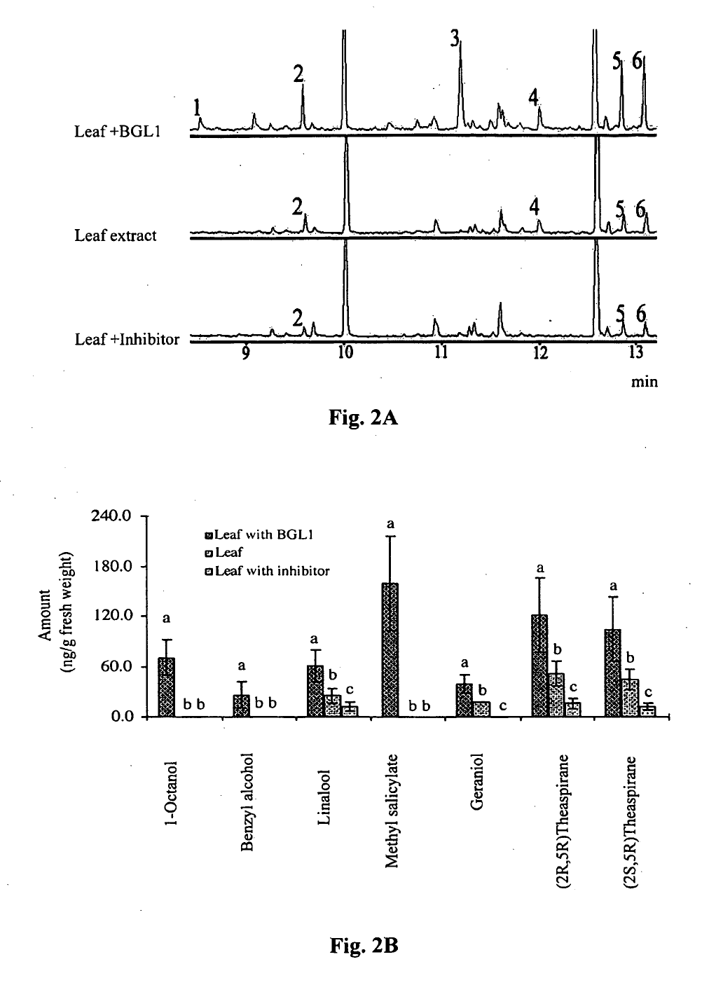

Headspace volatiles of the leaf extracts treated with BGL1, β-glucosidase inhibitor or nothing were collected with SPME fiber and analyzed by GC-MS. Identification and quantification of the detected volatiles were carried out by comparison of their mass spectra and retention indices with authentic synthetic standards. The levels of 1-octanol (CV, the coefficient of variation, 32.6%), linalool (28.6%), geraniol (22.4%), benzyl alcohol (32.1%), methyl salicylate (41.9%), (2R,5R)-theaspirane (38.7%) and (2S,5R)-theaspirane (32.4%) were significantly increased in the leaf extracts treated with BGL1 relative to leaf extracts treated with β-glucosidase inhibitor (FIG. 2), indicating that these compounds are present mainly as glucosides in apple leaves.

A. niger BGL1 released significantly more aglycones from leaf extract compared with leaf extract in the absence of exogenous β-glucosidase or β-glucosidase inhibitor. This may reflect either differences in the substrate specificity between A. niger BGL1 and apple β-glucosidase or simply higher activity of A. niger BGL1 in the reaction vial.

Most of the identified volatiles are considered common aglycones in many plant species (Stahl-Biskup et al., 1993). The data on apple aglycones are quite limited. A substantial amount of C13-noriprenoids has been found present as glycosidically bound aromatic compounds in apple fruit and leaves (Schwab and Schreier, 1988; Stingl et al., 2003. However, there was no report about geraniol, methyl salicylate, and theaspirane isomers as apple aglycones before.

The presence of certain aglycones may also vary between apple cultivars. Diastereomeric theaspiranes are found in nature (Schmidt et al., 1992). Two of them were identified as an aglycone in purple passion fruit (Passiflora edulis Sims) (Winterhalter, 1990). Schmidt et al. (1992) synthesized four isomers of theaspiranes that differed distinctly in their sensory properties. (2R,5R)-theaspirane was found with a weak camphoraceous note while (2S,5R)-theaspirane exhibited a strong camphoraceous, almost naphthalene-like note. In this study, the identification was based on these two synthetic theaspirane isomer standards. To the best of our knowledge, this is the first report of theaspirane isomers present as aglycones in apple leaves.

1-Octanol, benzyl alcohol, and methyl salicylate were detected only in the presence of A. niger BGL1. It might be because of the differences in substrate specificity of BGL1 and endogenous apple β-glucosidase or inefficient extraction of endogenous β-glucosidase. It is known that some plant glucosidases such as grape berry glucosidase are membrane-bound enzymes (Günata et al., 1998). Their extraction needs specific conditions. It is well documented that aglycone-moiety specificity of β-glucosidases varies considerably with the origin of the enzyme (Hösel and Conn, 1982; Günata et al., 1990; Babcock and Esen, 1994). In this study, low levels of linalool and theaspiranes were detected in leaf extracts treated with glucoimidazole, which completely inhibited β-glucosidase activity in the condition tested, suggesting that free forms of these compounds exist in apple leaves. Bengtsson et al. (2001) also detected a small amount of free linalool in the headspace of “Jonathan” apple leaves, but they did not mention theaspiranes.

It should be kept in mind that in addition to those above-mentioned compounds, there were many other volatiles released from apple leaf extract. Because of the limited sensitivity of the GC-MS, it is possible that there could be some other insect attractants or repellents present in the apple leaf head-space.

### Example 3

### Attractive/Repulsive Effect of β-Glucosidase-Enhanced Compounds on Codling Moth Insects

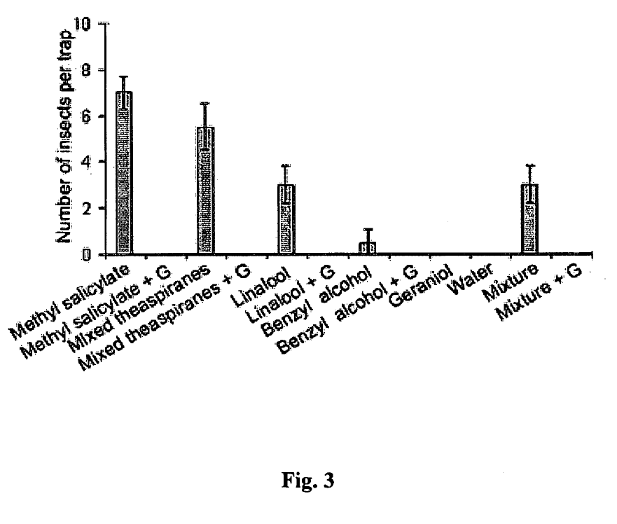

In cage bioassays, traps with each of the individual compounds at the concentrations detected in the leaf extracts were paired with a control trap containing only water and ethanol. The attractiveness of the compounds to the adult insects followed the decreasing order: methyl salicylate and a mixture of two theaspirane isomers, followed by linalool and then benzyl alcohol. All these compounds were more attractive than water as blank (FIG. 3). Interestingly, the trap with the mixture of all these compounds did not catch the most insects. To our knowledge, this is the first report of theaspirane isomers as a codling moth attractant. These compounds may be used as baits in mass-trapping of adult insects, in addition to the widely accepted technique of insect-mating disruption (Calkins and Faust, 2003).

The trap with 70 ng m⁻¹ 1-octanol did not catch any insects and the addition of the same amount of 1-octanol to any other compounds did not significantly affect the number of trapped insects compared with these compounds alone (data not shown). This indicated that 1-octanol does not have a significant effect on the behavior of codling moth adults.

Remarkably, 39.4 ng ml⁻¹ geraniol exhibited a repellent effect on codling moth adults. The traps with any of the aforementioned attractant compounds plus the same amount of geraniol did not capture any insects, suggesting that geraniol at that concentration eliminates the attractiveness of those compounds used in the experiments. Geraniol has been reported as a key ingredient in some commercial mosquito-repellent products (Xue et al., 2003) and as the main component of the natural essential oil of citrosa plants (Matsuda et al., 1996), which has been marketed as a biological repellent against mosquitoes.

Insect control focuses on both the protection of crops and animals and the maintenance of public health. One area of interest involves the development and production of environmentally safe and non-toxic insect repellents. Our results indicate that in the apple plant hosts of codling moth, geraniol exists as an inactivated glucoside/codling moth repellent. Only upon leaf injury does decompartmentation of the glucoside and β-glucosidase take place, resulting in the release of geraniol. More recently, we have shown that expression of the A. niger β-glucosidase gene (BGL1) in transgenic tobacco results in significant alteration of the volatiles in both intact and crushed leaves (Wei et al., 2004).

### Example 4

### Microcapsules Comprising Geraniol

Microcapsules comprising geraniol were prepared by first absorbing liquid geraniol in tricalcium phosphate (TCP) and then coating with the materials in lines 1-3 and 5 of Table 1, or as described in line 4 of Table 1:

TABLE 1
Microcapsules comprising geraniol

| | Absorbing | | Particle | | Geraniol |
|---|---|---|---|---|---|
| | carrier | Coating material | size | Appearance | content |
| 1 | Tri Calcium | Monoglycerides 60% | <850 μm | Free flowing | 15% |
| | Phosphate 25% | | | granular | |
| | | | | powder | |
| 2 | Tri Calcium | Ethyl Cellulose | <850 μm | Free flowing | 30% |
| | Phosphate 50% | CAS 9004-57-3 20% | | granular | |
| | | (Cellulose ethyl ether) | | powder | |
| 3 | Tri Calcium | Ethyl Cellulose + | <850 μm | Free flowing | 16% |
| | Phosphate 64% | Hydroxypropyl Cellulose | | granular | |
| | | 20% | | powder | |
| 4 | Matrix (filler) | A solution based on gelatin | <850 μm | Free flowing | 10% |
| | based on corn | is poured onto matrix and | | granular | |
| | starch 40% | the geraniol is added to | | powder | |
| | | create the beadlets 50% | | | |
| 5 | Tri Calcium | Ethyl Cellulose + | <850 μm | Free flowing | 16% |
| | Phosphate 54% | Hydroxypropyl Cellulose | | granular | |
| | | 30% | | powder | |

### REFERENCES

Adams, R. P. Identification of Essential Oil Components by Gas Chromatography/Mass Spectroscopy. Allured Publishing, Carol Stream, Ill., 1995.

Babcock, G. W.; Esen, A. Substrate specificity of maize β-glucosidase. Plant Sci. 1994, 101, 31-39.

Belancic, A.; Gunata, Z.; Vallier, M. J.; Agosin, E. Beta-glucosidase from the grape native yeast Debaryomyces vanrijiae: purification, characterization, and its effect on monoterpene content of a Muscat grape juice. J. Agric. Food Chem. 2003, 51, 1453-1459.

Bengtsson, M.; Backman, A. C.; Liblikas, I.; Ramirez, M. I.; Borg-Karlson, A. K.; Ansebo, L.; Anderson, P.; Lofqvist, J.; Witzgall, P. Plant odor analysis of apple: antennal response of codling moth females to apple volatiles during phenological development. J. Agric. Food Chem. 2001, 49, 3736-3741.

Calkins, C. O.; Faust, R. J. Overview of area wide programs and the program for suppression of codling moth in the western USA directed by the United States Department of Agriculture-Agricultural Research Service. Pest Manag. Sci. 2003, 59, 601-604.

Dan, S.; Marton, I.; Dekel, M.; Bravdo, B. A.; He, S.; Wither, S. G.; Shoseyov, O. Cloning, expression, characterization, and nucleophile identification of family 3, Aspergillus niger β-glucosidase. J. Biol. Chem. 2000, 275, 4973-4980.

Günata, Z.; Blondeel, C.; Vallier, M. J.; Lepoutre, J. P.; Sapis, J. C.; Watanabe, N. An endoglycosidase from grape berry skin of cv. M. Alexandria hydrolyzing potentially aromatic disaccharide glycosides. J. Agric. Food Chem. 1998, 46, 2748-2753.

Gunata, Z.; Bayonove, C. L.; Tapiero, C.; Cordonnier, R. E. Hydrolysis of grape monoterpenyl β-D-glucosides by various β-glucosidase. J. Agric. Food Chem. 1990, 38, 1232-1236.

Heightman, T. D.; Vasella, A. T. Recent insights into inhibition, structure, and mechanism of configuration-retaining glycosidases. Angew Chem. Int. Ed. 1999, 38, 750-770.

Hösel, W.; Conn, E. E. The aglycone specificity of plant β-glucosidase. Trends Biochem. Sci. 1982, 7, 219-221.

Knight, A. L.; Light, D. M. Attractants from Bartlett pear for codling moth, Cydia pomonella (L.), larvae. Naturwissenschaften 2001, 88, 339-342.

Landolt, P. J.; Brumley, J. A.; Smithhisler, C. L.; Biddick, L. L.; Hofstetter, R. W. Apple fruit infested with codling moth are more attractive to neonate codling moth larvae and possess increased amounts of (E,E)-alpha farnesene. J. Chem. Ecol. 2000, 26, 1685-1699.

Light, D. M.; Knight, A. L.; Henrick, C. A.; Rajapaska, D.; Lingren, B.; Dickens, J. C.; Reynolds, K. M.; Buttery, R. G.; Merrill, G.; Roitman, J.; Campbell, B. C. A pear-derived kairomone with pheromonal potency that attracts male and female codling moth, Cydia pomonella (L.) Naturwissenschaften 2001, 88, 333-338.

Matsuda, B. M.; Surgeoner, G. A.; Heal, J. D.; Tucker, A. O.; Maciarello, M. J. Essential oil analysis and field evaluation of the citrosa plant “Pelargonium citrosum” as a repellent against populations of Aedes mosquitoes. J. Am. Mosq. Control Assoc. 1996, 12, 69-74.

McLafferty, F. W. Wiley Registry of Mass Spectral Data, 6^{th} edition, J. Weiley & Sons, Inc., New York, N.Y., 1994.

Reed, H. C.; Landolt, P. J. Attraction of mated female codling moths (Lepidoptera: Tortricidae) to apples and apple odor in a flight tunnel. Florida Entomologist 2002, 85, 324-329.

Schmidt, G.; Full, G.; Winterhalter, P.; Schreier, P. Synthesis and enantiodifferentiation of isomeric theaspiranes. J. Agric. Food Chem. 1992, 40, 1188-1191.

Schwab, W.; Schreier, P. Simultaneous enzyme catalysis extraction: a versatile technique for the study of flavor precursors. J. Agric. Food Chem. 1988, 36, 1238-1242.

Shalit, M.; Guterman, I.; Volpin, H.; Bar, E.; Tamari, T.; Menda, N.; Adam, Z.; Zamir, D.; Vainstein, A.; Weis, D.; Pichersky, E.; Lewinsohn, E. Volatile ester formation in roses. Identification of an acetyl-coenzyme A geraniol/citronellol acetyltransferase in developing rose petals. Plant Physiol. 2003, 131, 1868-1876.

Shel'deshova, G. G. Ecological factors determining distribution of the codling moth Laspeyresia pomonella L. (Lepidoptera: Tortricidae) in the northern and southern hemisphere. Entomol. Rev. 1967, 46, 349-361.

Shoseyov, O.; Bravdo, B. A.; Siegel, D.; Ikan, R.; Goldman, A.; Cohen, S.; Shoseyov, L. Immobilized endo-β-glucosidase enriches flavor of wine and passion fruit juice. J. Agric. Food Chem. 1990, 39, 1387-1390.

Stahl-Biskup, E.; Holthuijzen, F. I. J.; Stengele, M.; Schulz, G. Glycosidically bound volatiles—a review 1986-1991. Flav. Fragr. J. 1993, 8, 61-80.

Stingl, C.; Knapp, H.; Winterhalter, P. 3,4-Dihydroxy-7,8-dihydro-beta-ionone 3-O-beta-D-glucopyranoside and other glycosidic constituents from apple leaves. Nat. Prod. Lett. 2002, 16, 87-93.

Sutherland, O. R. W.; Hutchins, R. F. N. α-Farnesene, a natural attractant for codling moth larvae. Nature 1972, 239, 170.

Wearing, C. H.; Hutchins, R. F. N. α-Famesene, a naturally occurring oviposition stimulant for the codling moth, Laspeyresia pomonella. J. Insect Physiol. 1973, 19, 1251-1256.

Wei, S.; Marton, I.; Dekel. M.; Shalitin, D.; Lewinsohn, E.; Bravdo, B. A.; Shoseyov, O. Manipulating volatile emission in tobacco leaves by expressing Aspergillus niger β-glucosidase in different subcellular compartments. Plant Biotechnol. J. 2004, 2, 341-350.

Winterhalter, P. Bound terpenoids in the juice of the Purple passion fruit (Passiflora edulis Sims). J. Agric. Food Chem. 1990, 38, 452-455.

Xue, R. D.; Ali, A.; Barnard, D. R. Laboratory evaluation of toxicity of 16 insect repellents in aerosol sprays to adult mosquitoes. J. Am. Mosq. Control Assoc. 2003, 19, 271-274.

Yan. F.; Bengtsson, M.; Witzgall, P. Behavioral response of female codling moths, Cydia pomonella, to apple volatiles. J. Chem. Ecol. 1999, 25, 1343-1351.

Yanai, T.; Sato, M. Isolation and properties of β-glucosidase produced by Debaryomyces hansenii and its application in winemaking. Am. J. Enol. Vitic. 1999, 50, 231-235.

Zhu, J.; Park, K. C.; Baker, T. C. Identification of odors from overripe mango that attract vinegar flies, Drosophila melanogaster. J Chem. Ecol. 2003, 29, 899-909

1. A composition comprising geraniol for use as codling moth repellent.

2. The composition according to claim 1 wherein the geraniol is in mixture with further ingredients selected from the group consisting of nerol and citronellol.

3. The composition according to claim 2 comprising from about 50 up to about 70% by weight of geraniol, from 0 up to about 20% by weight of nerol, and from about 20 up to about 40% by weight of citronellol.

4. The composition according to claim 1 wherein the geraniol or geraniol mixture is contained in a polymer matrix.

5. The composition according to claim 1 in sprayable form.

6. The composition according to claim 1 provided as a concentrate for dilution, dispersion or dissolution in water to provide a sprayable composition.

7. The composition according to claim 1 comprising microcapsules containing geraniol or the geraniol mixture.

8. The composition according to claim 7 for slow release of the geraniol or geraniol mixture.

9. A method for repelling codling moth which comprises treating an environment infested or imminently capable of being infested with said codling moth with a codling moth repelling composition comprising geraniol.

10. The method according to claim 9 wherein said composition comprises a geraniol mixture with further ingredients selected from the group consisting of nerol and citronellol.

11. The method according to claim 9 wherein said composition is a sprayable composition comprising microcapsules containing geraniol or a geraniol mixture.

12. The method according to claim 9 wherein said environment is a fruit orchard including a pear, apple, quince, walnut, hawthorn and crab apple orchard.

13. The method according to claim 12 wherein said fruit orchard is an apple orchard.

14. The method according to claim 12 wherein the composition is sprayed in said fruit orchard.

15. A method of repelling codling moth in apple orchards, said method comprising treating the apple orchards with a geraniol repellent composition.

16. A composition comprising an agent selected from the group consisting of methyl salicylate, a theaspirane isomer, linalool, benzyl alcohol, and a combination thereof, for use as codling moth attractant.

17. The composition according to claim 16, wherein said agent is methyl salicylate, (2R,5R) theaspirane, (2S,5R) theaspirane, or a combination thereof.

18. The composition according to claim 16 wherein the attractant or combination thereof is contained in a polymer matrix.

19. The composition according to claim 16 in sprayable form.

20. The composition according to claim 16 provided as a concentrate for dilution, dispersion or dissolution in water to provide a sprayable composition.

21. The composition according to claim 16 in the form of microcapsules.

22. The composition according to claim 21 for slow release of the codling moth attractant or combination of attractants.

23. A method for attracting codling moth to an insect trap, which comprises treating an environment surrounding said trap infested or imminently capable of being infested with codling moth, with a codling moth attractant selected from the group consisting of methyl salicylate, a theaspirane isomer, linalool, benzyl alcohol, and a combination thereof.

24. The method according to claim 23, wherein said environment is a fruit orchard including a pear, apple, quince, walnut, hawthorn and crab apple orchard.

25. The method according to claim 24 wherein said fruit orchard is an apple orchard.

26. A method for integrated control of codling moth in fruit orchards which comprises treating an environment of the orchard infested or imminently capable of being infested with codling moth with a codling moth repelling composition comprising geraniol and a codling moth attractant composition comprising an attractant selected from the group consisting of methyl salicylate, a theaspirane isomer, linalool, benzyl alcohol, and a combination thereof.

27. A method for identifying volatile insect repellents and/or attractants released from their non-volatile glucosides by the action of β-glucosidase on plant tissue extract, comprising:
(i) homogenizing plant tissue and subjecting the plant tissue homogenate to the action of an extraction buffer;
(ii) treating the plant extract with a β-glucosidase or with a β-glucosidase inhibitor;
(iii) identifying by GC-MS specific volatiles released in step (ii) which levels were increased in the extracts treated with β-glucosidase relative to the extracts treated with the β-glucosidase inhibitor;
(iv) testing each one of the volatiles identified in step (iii) for their effect on the behavior of insects, thus identifying volatiles which are repellents or attractants of each of the tested insects.

28. A method according to claim 27 wherein the β-glucosidase is a recombinant β-glucosidase and the β-glucosidase inhibitor is glucoimidazole.
